(19) United States
(12) Patent Application Publication
(10) Pub. No.: US 2018/0028111 A1
(43) Pub. Date: Feb. 1, 2018

(54) FAMILY SLEEP MONITORING SYSTEM

(71) Applicant: Apple Inc., Cupertino, CA (US)

(72) Inventors: Mikko WARIS, Helsinki (FI); Helena WARIS, Helsinki (FI); Joonas PAALASMAA, Helsinki (FI)

(21) Appl. No.: 15/548,744

(22) PCT Filed: Jan. 28, 2016
(86) PCT No.: PCT/FI2016/050048

§ 371 Date: Aug. 3, 2017

Related U.S. Application Data
(60) Provisional application No. 62/111,170, filed on Feb. 3, 2015.

(51) Int. Cl. A61B 5/00; A61B 5/16

(52) U.S. Cl. CPC A61B 5/4815; A61B 5/165; A61B 5/0022; A61B 2503/06; A61B 2505/07

(57) ABSTRACT

A distributed computer system 100 and method that will serve for recording sleep of at least one user using a mobile subscriber terminal 104 and a peripheral device 102 arranged to communicate with the mobile subscriber terminal 104. The present invention records the sleep parameters of family members by providing third party applications and a plurality of peripheral devices communicating with the mobile subscriber terminals according to the age and interest of each family member. The peripheral devices are configured to communicate sleep data to the mobile subscriber terminals and further to a data storage application 120 stored on a server computer 130. For a child user the peripheral device 102 and mobile subscriber terminal 104 may be incorporated into a soft toy 110.

### FIELD OF INVENTION

The present invention relates to a system for recording sleep of at least one user. More specifically, the present invention is related to a system for measuring sleep of a family using a plurality of peripheral devices according to age and interest of each family member.

### BACKGROUND OF INVENTION

Sleep is an important parameter for the wellbeing of individuals and sleeping problems of a family member often affect other members of the family. Generally, it has been seen that if a child has sleep problems, the whole family is often affected during both night and day. If child sleep problems are not addressed properly, they can persist and can be linked to a variety of developmental and behavioral problems. Therefore recognition of sleep problems at an early stage of a child's life is necessary and has great benefits for the child and for rest of the family.

A few systems are known in the prior art for monitoring sleep of a user, for example, the ‘Beddit’ app and sleep measurement system. The Beddit App provides a sleep score using a Beddit sensor that is installed to user's bed. This app is publicly available on the Apple iOS App Store. Further, there are more applications available for tracking the growth of infants, children, and adolescents using pediatric growth charts, for example, “Pediatric Growth Charts” by Ossus GmbH. The iTunes excerpt is cited here as reference.

Further, a patent application WO2014/164568 discloses a method and a system for treatment of sleep and growth disorders. The patent application provides a medical method for treating human pediatric growth hormone deficiency (PGHD) in a pediatric patient. This document is hereby cited as reference in this application.

There is also disclosed in a patent application US 201110021188 A1, a communication system that comprises a mobile phone of a mother capable of regularly reporting newborn data such as body temperature, defecation, urination, breast-feed and disgorgement by means of infrared communicator with radio wave telephone communicator disabled. The hospital has a receiving system for processing the report for administrating the newborn. This document is hereby cited as reference in this application.

However, despite of the advancement offered by the techniques disclosed in the prior art, a need remains for further improvements, particularly with regard to the manner in which the sleep of one or more persons of different age groups can be monitored simultaneously. Most of the systems available in the prior art are featured only for a particular age group. They are not efficient for analyzing sleep related data of people in different age groups. Further, many sensor peripherals used in such devices are not child friendly, for example, children do not like to use wired devices.

Many sleep problems in human beings can be addressed by study of human behavior, and the quality of sleep can be improved by setting and enforcing proper sleep times, ensuring a good sleep environment and timely parent contact.

Hence, in light of the above discussion, there is a need for devising a system for measuring sleep of all family members, especially of children, by providing customized peripherals and third party applications according to age and interest of each member.

### SUMMARY OF INVENTION

The invention under study is directed towards a system and a method for effectively monitoring the sleep of at least one user.

The present invention discloses a distributed computer system comprising at least one server computer; at least one mobile subscriber terminal arranged to be in a communication connection with a data storage application on said server computer for the non-volatile storage of data; and at least one peripheral device. The peripheral device is arranged to measure sleep data of the at least one user and to communicate said sleep data via radio path to a third party application configured to be executed on said mobile subscriber terminal. Further, the third party application is arranged to communicate the sleep data into the non-volatile storage of data in said at least one server.

It is a further object of the present invention to present a distributed computer system that includes a plurality of peripheral devices arranged to measure one or more users belonging to a multi-person family.

It is another object of the present invention to present a distributed computer system to aggregate the sleep data from all measured family members on a server computer. In one aspect of the present invention, the aggregated sleep data of one or more users is arranged to be displayed together to visualize how the sleeping behaviors of the family members influence each other.

It is yet a further object of the present invention to present a method for recording the sleep of at least one user. The method includes the following steps: Initially a communication connection is implemented between a mobile subscriber terminal and a data storage application stored on a server computer. In next step, at least one peripheral device is arranged from a plurality of peripheral devices for measuring the sleep of at least one user belonging to a multi-person family. Further, the peripheral device communicates the sleep data to a third party application executing on a mobile subscriber terminal. Thereafter, the third party application communicates the sleep data to a non-volatile storage of data in said at least one server computer. In next step, the sleep data of one or more users belonging on the multi-person family is aggregated to said server computer. Further, the sleep data of a family of users is displayed to visualize how the sleeping behavior of an individual user influences other family members.

It is another object of the present invention to provide a computer program product for recording the sleep of at least one user by a computing device, comprising at least one non-transitory computer-readable storage medium having computer-readable program code portions stored therein. The computer-readable program code portions including instructions are arranged to implement the aforementioned method of recording the sleep of at least one user.

A distributed computer system for measuring sleep of at least one user is in accordance with the invention, the distributed computer system comprising
at least one server computer;
at least one mobile subscriber terminal arranged to be in a communication connection with a data storage application on said server computer for the non-volatile storage of data;
at least one peripheral device; wherein at least one peripheral device is arranged to measure sleep data of the at least one user belonging to a multi-person family and to communicate said sleep data via radio path to a third party application configured to be executed on said mobile subscriber terminal, further the third party application is arranged to communicate the sleep data to the data storage application executing on the server computer;
   further wherein the sleep data of at least one measured users is arranged to be aggregated to said server computer, and the aggregated sleep data is arranged to be displayed together to visualize how sleeping behavior of individual user belonging to the multi-person family influences other users of the same multi-person family.

A method of recording sleep data of at least one user is in accordance with the invention the method comprising:
implementing a communication connection between a mobile subscriber terminal and a data storage application stored on a server computer;
arranging at least one peripheral device from a plurality of peripheral devices to measure the sleep of at least one user belonging to a multi-person family;
communicating the sleep data via radio path to a third party application executing on a mobile subscriber terminal;
communicating the sleep data from the third party application to a non-volatile storage of data in said at least one server computer;
arranging to aggregate the sleep data of one or more users belonging to the multi-person family to said server computer, and
displaying the sleep data of one or more users belonging to the multi-person family to visualize how the sleeping behaviors of the individual user belonging to the multi-person family influence other users of the same multi-person family.

A computer program product arranged to measure sleep data of at least one user is in accordance with the invention, the computer program product comprising at least one non-transitory computer-readable storage medium having computer-readable program code portions stored therein, said computer-readable program code portions comprising instructions:
implementing a communication connection between a mobile subscriber terminal and a data storage application stored on a server computer;
arranging at least one peripheral device from a plurality of peripheral devices to measure the sleep of at least one user belonging to a multi-person family;
communicating the sleep data via radio path to a third party application executing on a mobile subscriber terminal;
communicating the sleep data from the third party application to a non-volatile storage of data in said at least one server computer;
arranging to aggregate the sleep data of one or more users belonging to the multi-person family to said server computer.

The distributed computer system, the method and the program product mentioned before may involve displaying the sleep data of one or more users belonging to the multi-person family to visualize how the sleeping behaviors of the individual user belonging to the multi-person family influence other users of the same multi-person family in accordance with the invention.

The distributed computer system, the method and the program product mentioned before may involve an embodiment where there is no server and all computing takes place at a mobile subscriber terminal in accordance with the invention.

The distributed computer system, the method and the program product mentioned before may also involve an embodiment where the peripheral device communicates directly with the server computer, and there is no mobile subscriber terminal in the implementation in accordance with the invention.

In an embodiment of the present invention, the peripheral device is incorporated into a soft toy to be used by a child user. In another embodiment of the present invention the peripheral device is placed in a sensor box wrapped with a plastic sheet having cartoon characters embossed on it. This may attract the child user to play with the cartoon characters and keep the sensor box with him while sleeping. The sensor box can measure the sleep of the child user and can communicate the sleep data to the third party application executing on a mobile subscriber terminal and/or data storage application executing on the server computer.

In another embodiment of the present invention, a virtual pet application is utilized to encourage the child user to follow sleep guidelines. The virtual pet application gives rewards to the child user for following a proper time schedule. The rewards can be in form of virtual currency to buy in-app donuts, cookies etc. for the virtual pet. In some embodiments, the third party application is arranged to communicate with the virtual pet application. The third party application can provide the sleep results to the virtual pet application and in case of good sleep results; the third party application can instruct the virtual pet application to give rewards to encourage the child user to follow the sleep schedule properly assigned to him by the parent user.

Some or all of the aforementioned advantages of the invention are accrued by a distributed computer system for tracking sleep of the all family members by providing soft toy and/or virtual pet to the child user and wellness information to adults using the server based data to improve the health and wellness over time, and hence change sleeping behavior of one or more users.

In addition and with reference to the aforementioned advantage accruing embodiments, the best mode of the invention is considered to be where the computer peripheral is realized in a soft toy that is used by a child. The soft toy is arranged to measure the sleep of at least one child and in which the sleeping data can be seen by e.g. turning the eyes of the toy. Alternatively and in case of older children the third party software application communicates with a virtual pet software application, which allows the child user in app purchases. The virtual pet motivates the child to use the peripheral device and the system in a way that maximizes healthy sleeping habits for the child.

In some embodiments, the distributed computer system comprises a plurality of applications. The plurality of applications includes one or more of following: one or more third party applications, soft toy application, calendar application and data storage application. The distributed computer system is enabled by the user to visualize the sleep data of a long time frame such as of years, months, weeks and days.

The third party application, such as Beddit app is capable of transforming the sleep data into human readable format. The peripheral device is basically a sensor that senses the sleeping conditions and parameters of a user such as whether room light is on or off, user presence on the bed, user presence in the room, user's sleep time, user's awake time, user's physical parameters (height, weight, temperature, blood pressure, respiration etc.) and user's physiological parameters (anxiety, depression, happy, sad, sick etc.). The third party application may analyze the data sensed by the peripheral device and may further communicate the analyzed data with the calendar and/or data storage application. The calendar application may enable the data to be visualized in the time view such as month view, year view, and in other ranges. The data storage application may be arranged to aggregate the sleep data of one or more users belonging to the multi-person family on the server computer. The data storage application further provides the comparison of sleep data of individual user with the other users of the same multi-person family to visualize the influence of sleep behavior on each other.

### BRIEF DESCRIPTION OF THE DRAWINGS

In the following the invention will be described in greater detail with reference to exemplary embodiments in accordance with the accompanying drawings, in which:

FIG. 1 demonstrates an embodiment 10 of the distributed computer system 100 comprising a server computer 130 in network 128 with the mobile subscriber terminals 104, 108 and the peripheral devices 102, 106 for a three person family in accordance with the invention.

FIG. 2 demonstrates an embodiment 20 where a soft toy 110 is directly in connection with the server computer 130 via internet in accordance with the invention.

FIG. 3 demonstrates a sleep recording method in an embodiment 30 in accordance with the invention.

FIG. 4 demonstrates an embodiment 40 of the data storage application 120 and/or the third party application 122 providing sleep data in accordance with the invention.

FIG. 5 demonstrates an embodiment 50 providing sleep scores for an exemplary family in accordance with the invention.

FIG. 6 demonstrates an embodiment 60 providing a diagnosis of the long term sleep history of the user in accordance with the invention.

FIG. 7 demonstrates an embodiment 70 providing the statistics and the sleep data of one user being analyzed against macro population demographics in accordance with the invention.

FIG. 8 demonstrates a soft toy 110 embodiment 80 with plastic/fabric cover 800 on the sensor box 810 in accordance with the invention.

FIG. 9 demonstrates a virtual pet application 900 embodiment 90 with an in-app purchase facility in accordance with the invention.

FIGS. 10-15 comprise description of an actual product based on the invention, which will soon be launched after the filing of the present application.

FIGS. 16-20 comprise screenshots of the actual product in secret beta-testing phase.

FIG. 21 demonstrates the unpublished press release describing one embodiment of the product of the invention.

The subject matter of the preceding drawings is also explicitly incorporated into the description. To remove any doubt the Applicant makes his intention to claim priority and support from the preceding figures hereby explicit.

The features of the invention illustrated above and below in the specification, are described with reference to the drawings summarized above. The reference numbers shown in the drawings may be used at one or more places to indicate the functional relation between the referenced elements. Some of the embodiments are described in the dependent claims.

### DETAILED DESCRIPTION OF THE EMBODIMENTS

The objects of the present invention are accomplished, and the problems and shortcomings associated with the prior art techniques and approaches are overcome, by the present invention as described below in the preferred embodiments.

Embodiments of the present invention provide a distributed computer system 100 for measuring sleep of the users belonging to a multi-person family. The distributed computer system 100 includes a plurality of peripheral devices, one or more mobile subscriber terminals and a server computer. The distributed computer system 100 further may include a family of applications. The family of applications includes one or more of following: one or more third party applications, soft toy application, calendar application and data storage application.

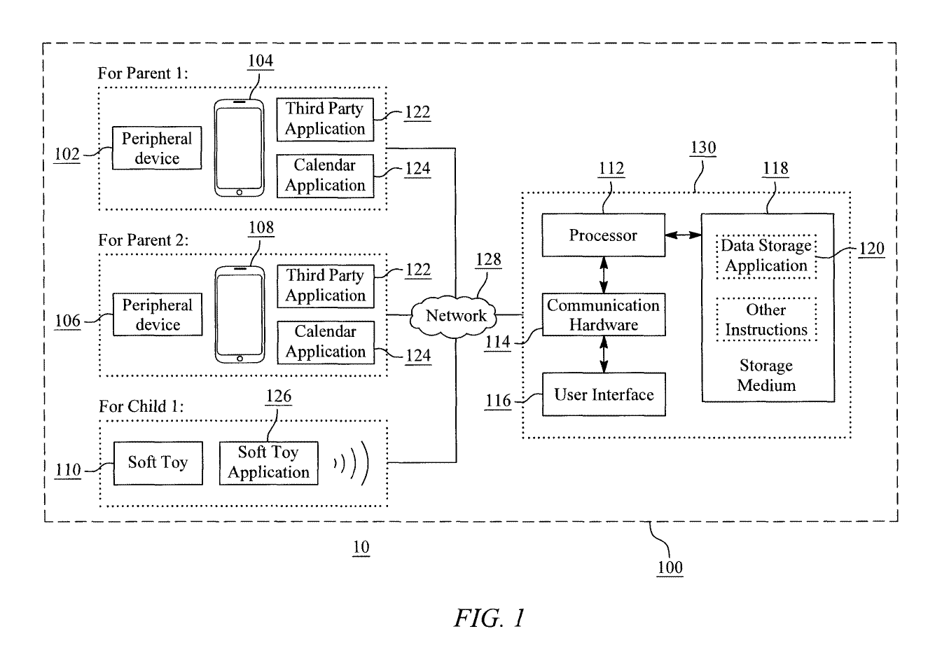

Referring to FIG. 1, an embodiment 10 of the present invention provides a distributed computer system 100 for measuring sleep of three users of a multi-person family. The users, for example, in this embodiment are a parent 1, a parent 2 and a child 1. In the embodiment, a peripheral device and a mobile subscriber terminal are provided to each adult user. The parent 1 is provided with a peripheral device 102 and mobile subscriber terminal 104 and the parent 2 is provided with a peripheral device 106 and mobile subscriber terminal 108. The peripheral devices 102, 106 may be incorporated in an object such as a wrist watch, a smart phone, a smart watch, a bracelet, a bed-mattress, a wallet etc. The peripheral devices 102, 106 measure the sleep data of parent 1 and parent 2 respectively and further communicate the sleep data to the mobile subscriber terminals 104, 108.

In one aspect, the peripheral devices 102, 106 communicate the sleep data to the third party application 122 executing on the mobile subscriber terminals 104, 108 via a radio path. For communicating the sleep data any communication technology can be utilized such CDMA (Code Division Multiple Access), GSM (Global System for Telecommunications), UMTS (Universal Mobile Telecommunication System), HSPA (High Speed Packet Access), EV-DO, EV-DO rev. A, 3GPP LTE (3^{rd} Generation Partnership Project Long Term Evolution), WiMAX, Wi-Fi, Bluetooth, Low Energy Bluetooth, Internet, telephony, or other communication formats including combinations thereof.

The mobile subscriber terminals 104, 108 are further arranged to communicate with the server computer using network 124. Examples of the network 124 can include, but is not limited to, an internet network, a Bluetooth network, a radio telephone network or any other communication medium that may enable the communication between them. The third party application 122 further communicates the sleep data to a data storage application 120 executing on the server computer 130.

In an embodiment of the present invention, a soft toy 110 is configured to provide the sleep data of the child 1. A child user of age below 7 years generally does not have access to mobile subscriber terminals or may not be able to use a mobile subscriber terminal. Therefore, for such scenarios, the child user is provided with a soft toy peripheral in this embodiment. The soft toy includes an inbuilt peripheral device to measure the sleep of the child 1. Further, a soft toy application is provided 126 to communicate the sleep data of the child 1 to the data storage application 120 executing on a server computer 130.

The server computer 130 includes a processor 112, communication hardware 114 and user interface 116. The server computer 130 may be any device such as a mobile terminal, a dedicated device, computer, tablet, cloud server and/or centralized hubs of hospitals and clinics etc.

In the embodiment of the present invention, the family of application includes the third party applications 122, soft toy application 126, calendar application 124 and data storage application 120. In some embodiments, one or more applications from the family of applications are configured to communicate with each other.

The third party application 122 such as the Beddit app is capable of transforming the sleep data into human readable format.

The peripheral devices like the peripheral devices 102 and 106 or the soft toy 110 include a sensor that senses the sleeping conditions and parameters of a user such as room light is on or off, user presence on the bed e.g. by ballistocardiographic measurements, user presence in the room, user's sleep time, user awake time, user's physical parameters (height, weight, temperature, blood pressure, respiration etc.) and user's physiological parameters (anxiety, depression, happy, sad, sick etc.). The said at least one sensor may involve any of the following sensor technologies: photoelectric sensor, piezo thin film sensors, strain gauge, capacitive sensors, proximity sensors, thermocouples, inductive sensors and/or excited piezo thin film sensors individually or in any combination. The best mode of the inventive peripheral device is believed to be represented by an embodiment in which one or more thin film piezo sensors are integrated with a plethora of different types of static sensors, namely the strain gauge sensor and the capacitive load cell sensor and the capacitive proximity sensor and the thermocouple and the inductive proximity sensor. There may be a plurality or just a single sensor of each type in the best mode. The combination of thin film piezo sensors (dynamic measurement technique) with a plurality of different static measurement techniques is especially preferable. This is because the combination of static measurement (is the user weighing on the bed) when combined with dynamic (is there a human moving on the bed) allows the computer system to accurately determine when in fact the user starts to sleep, without receiving any input from the user itself. The application U.S. 62/087,870 of the Applicant discloses many sensor technologies developed by the Applicant, and the sensor solutions of this application are directly and explicitly incorporated into this application,

The third party application 122 can analyze the data sensed by the peripheral device and may further communicate the analyzed data with the calendar application 124 and/or data storage application 120. The calendar application 124 can enable the data to be visualized against time such as viewed over a month, or a year, or in any other custom ranges. The data storage application 120 is configured to aggregate the sleep data of the one or more users belonging to the multi-person family on the server computer 130. The data storage application 120 further provides the comparison of sleep data of one or more users to visualize the influence of one or more users on other family members.

In an embodiment, the plurality of applications is configured to show how the child sleeps compared to general guidelines of sleep, amount of sleep and awake time in bed. The applications are further enabled to check, typically via the peripheral device, whether the child is sleeping or doing something else such as playing games or listening to music etc. The family of applications is further enabled to deduce the stages of sleep of the sleeping user, for example, deep sleep, light sleep, active sleep stages etc. based on the sleep data.

In some embodiments of the present invention, the family of applications is arranged to analyze how the child's sleep evolves over time. The server computer 130 is arranged to record the weight and height of the child user as a function of time. Thus the server computer 130 stores and displays the analyzed data by depicting the relation between sleep and physical growth of the child user. Using the calendar application 124, the physical growth of the child user and the sleep data can be visualized in time mapped format. In an embodiment of the present invention, the visualization of the physical growth and the sleep data may be in the form of a growth chart that is generally used to track the weight and the height of the child. Further, the applications can also analyze how the child user's sleep patterns evolve from almost continuous sleep and no rhythm to the day/night rhythm of a small child. As the child user needs a longer sleeping time in comparison to the adult users, the third party application can help in determining how the amount of sleep needed decreases according to the age, but continues to be higher than that for adults.

In kindergarten/school age, the child user is required to wake up at a specified time typically early. In some embodiments of the invention, the third party application may analyze whether the child user is getting enough sleep, or how easy it is for the child user to fall asleep at evening, and wake up in the morning. Further, the third party application may also check if anyone in the family has issues with his/her sleep; or how a particular family member's sleep affects other family members; or does each family member sleep normal compared to recommendations, and whether each family member sleeps normally when compared to same age group and sex, or whether there is a change in sleep pattern with age etc.

The family of applications is further arranged to provide instructions for improving the sleeping conditions of each family member.

In one embodiment of the present invention, one peripheral device is arranged to communicate with the one dedicated mobile subscriber terminal, for example in the embodiment each user can have their personal peripheral devices and personal mobile subscriber terminals.

In a further embodiment of the present invention, multiple peripheral devices may be arranged to communicate with a single mobile subscriber terminal. In such an embodiment, each user may have separate peripheral devices and/or a plurality of peripheral devices that are arranged to communicate with a single dedicated mobile subscriber terminal. In the embodiment, all individual users belonging to the same multi-person family may have one or more peripheral devices that are arranged to communicate the sleep data to the third party application executing on the single mobile subscriber terminal. In the embodiment, the soft toy of the child user is also arranged to communicate the sleep data to the third party application executing on the same mobile subscriber terminal on which the parent users sleep data is being communicated.

A further embodiment of the present invention provides a distributed system in which multiple mobile subscriber terminals may be arranged to read the sleep data of a single peripheral device. For example, the peripheral device of a child user is arranged to communicate the sleep data to the mobile subscriber terminals of both the parent users and/or of a care taker.

It should be noted that any features, phases or parts of embodiment 10 can be freely permuted and combined with any of the embodiments 20, 30, 40, 50, 60, 70, 80, and/or 90 in a combination of two or more embodiments in accordance with the invention.

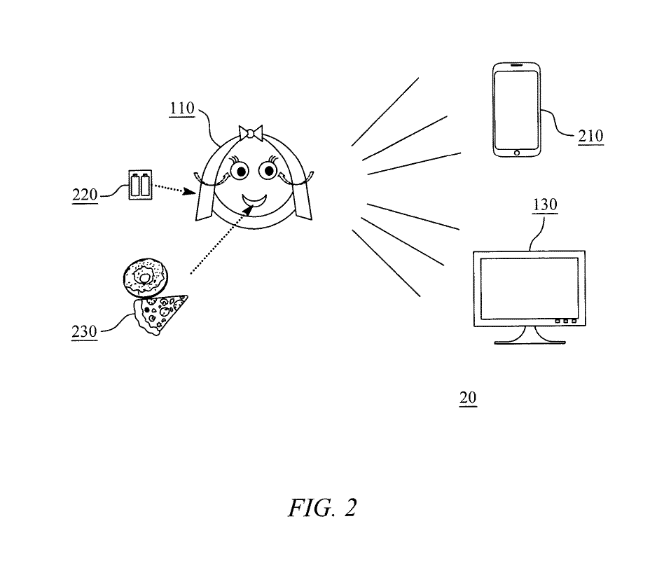

Referring to FIG. 2, an embodiment 20 of the present invention provides a soft toy 110 to be used by a child user. The soft toy 110 can comprise an inbuilt mobile subscriber terminal and a peripheral device. The inbuilt mobile subscriber terminal communicates the sleep data sensed by the peripheral device to the data storage application 120 of the server computer 130. In an embodiment of the present invention, the soft toy is made of soft cotton fur of vibrant color to fascinate the child user to keep the soft toy with him while sleeping.

In the embodiment of the present invention, the eyes of the soft toy 110 may include a display and a user interface to visualize the sleep data. In the embodiment, the eyes of the soft toy 110 may be turned around with a simple touch and the parent user may read the sleep data of child user by looking in to the eyes of the soft toy 110. In some embodiments of the present invention, the eyes of the soft toy 110 may be configured to recognize the parent or child user using one or more biometric recognition techniques such as finger print recognition, iris recognition etc.

In an embodiment of the present invention, the peripheral device incorporated in to a soft toy 110 may include a sleep sensor and a data storage unit. In the embodiment, the sleep sensor may be placed in belly portion of the soft toy 110 to measure the sleep data of the child user and the data storage unit may be place in the head portion of the soft toy 110.

The soft toy 110 works with a soft toy application 126 that can incorporate a plurality of third party applications including the calendar application 124.

In some embodiments of the present invention, the soft toy 110 may be operable using a battery 220. The battery 220 can be inserted in a battery cabinet in the soft toy 110, for example it can be on the backside of the soft toy 110. Further, in one embodiment, to make it more real-life, the child user can feed the soft toy 110. To enable this, a detachable food bag can be provided inside a mouth section of the soft toy 110. In the embodiment of the present invention, the child user can feed the soft toy 110 with cookies, donuts 230 etc. This encourages the child user to play, eat and sleep with the soft toy 110 and to treat the toy as a night buddy. In this way the child user will be in contact with soft toy 110 for longer duration of time, which makes reading sleep data easier and more accurate.

In an embodiment of the present invention, the mobile subscriber terminal of soft toy 110 is configured to communicate the sleep data directly to the mobile subscriber terminal 210 of the parents of the child user. The soft toy 110 can be any toy for e.g. a Barbie Doll®, an Angry Bird®, a Pou, a Nemo etc. In other embodiments of the present invention, the soft toy application 126 may communicate the sleep data directly to the data storage application 120 of the server computer 130. This feature of the invention is an advantage for infants and child users who cannot use a mobile subscriber terminal. The soft toy 110 is a wireless capable device and does not involve any material strips that would make it uncomfortable for the infant and/or child user during sleep recording.

In some embodiments of the present invention, the soft toy 110 can be embedded with a speaker and audio devices to make the child user fall asleep easily. The mobile subscriber terminal, peripheral device, speaker, audio device may be placed in a way that one or more of them can be removed so that the soft toy 110 can be washed. Further, in another embodiment of the present invention, the soft toy 110 can be in the form of a stuffed animal, ball, a soft/fabric book, or other soft toys for infants. In some embodiments of the present invention, the soft toy 110 may be in the form of an infant toy such as a milk bottle, a lunch box, a pencil box, a music system or other toys that may be placed on a child's bed or other locations near the child's place of sleep.

In some embodiments, the soft toy may include a camera and audio and/or visual alert devices to monitor the activities of the child user and to alert the parents of the child user from time to time. The periodic alert can also be configured using one or more third party applications for alerting the child user's parent from time to time.

In other embodiments of the present invention, the soft toy can be implemented in shape of Angry Birds® that may have LED eyes for functioning as a lamp in night time. The LED eyes may be automatically operable and may provide the sleep data by rotating/turning the eyes around. Using the soft toys like angry bird as a sleep peripheral for measuring sleep would be comfortable and exciting for the kid users.

It should be noted that any features, phases or parts of embodiment 20 can be freely permuted and combined with any of the embodiments 20, 30, 40, 50, 60, 70, 80, and/or 90 in a combination of two or more embodiments in accordance with the invention.

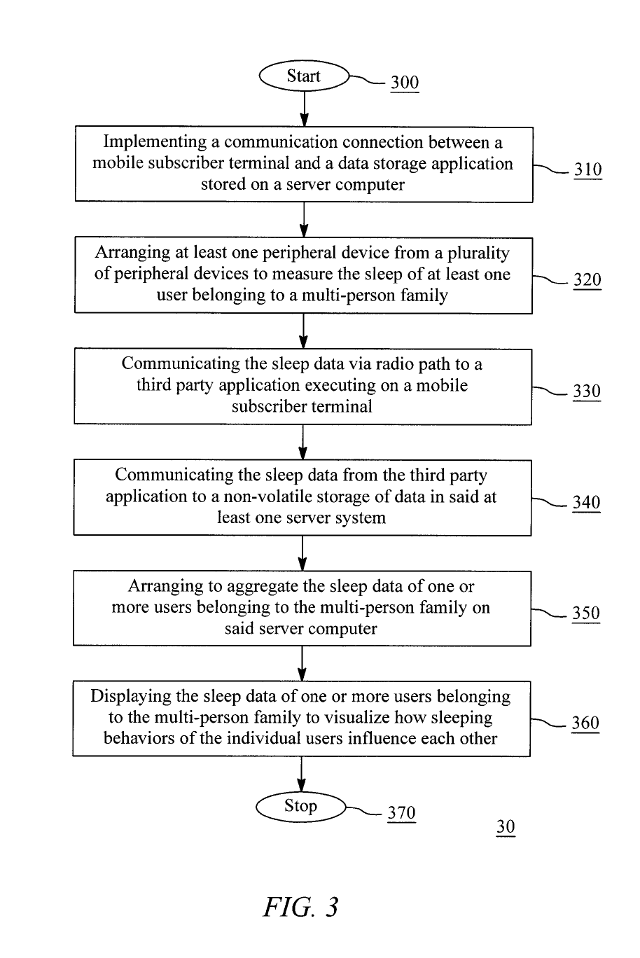

As depicted in FIG. 3, another embodiment 30 of the present invention provides a sleep recording method. The method is initiated at step 300.

At step 310, the distributed computer system 100 implements a communication connection between a mobile subscriber terminal, for example the mobile subscriber terminal 104 and the data storage application 120 stored on the server computer 130.

Further, at step 320, at least one peripheral device from a plurality of peripheral devices is arranged to measure sleep of at least one user belonging to a multi-person family. The peripheral device is a sensor device that can include one or more sensors to measure the sleep of the user.

Thereafter at step 330, the peripheral device communicates the sleep data via the radio path to a third party application executing on the mobile subscriber terminal. For communicating the sleep data any communication technology can be utilized such as CDMA, GSM, UMTS, HSPA, EV-DO, EV-DO rev. A, 3GPP LTE, WiMAX, Wi-Fi, Bluetooth, Low Energy Bluetooth, Internet, telephony, or other communication formats including combinations thereof.

The sleep data may include one or more of following such as sleep time, awake time, user presence on bed, time taken to fall asleep after going to bed, data of activities before falling sleep (playing games, watching movie, listening songs etc.), ambient conditions (illumination intensity, noise level, room temperature etc.), health related data (respiration, heart rate, blood pressure etc.), physiological data (anxiety, depression, insomnia etc.) and growth data (height, weight, Body mass index etc.)

Further at step 340, the measured sleep data is communicated to the data storage application 120 from the third party application. The data storage application 120 stores the sleep data, typically as a function of time.

Subsequently at step 350, the sleep data of the one or more users belonging to the multi-person family is arranged to be aggregated on the server computer 130.

At step 360, the sleep data of the one or more users belonging to the multi-person family is displayed to visualize how the sleeping behaviours of the one or more users belonging to the multi-person family influence each other.

The sleep behaviour can be visualized by the third party and/or data storage application by collaborating and comparing the sleep data of individual users with other users of belonging to the same multi-person family and/or other users of the age of individual users. Often, it has been seen that if the child user is having some problem such as insomnia, fever etc.; at least one parent user's sleep is also disturbed. This can be seen by analysing the sleep data of two users as a function of time. For example, if a child user starts crying at 11 p.m. and the mother parent user wakes up at 11.05 p.m., it may be visualized on the server computer how much of the time of the mother parent user is affected, in which state of sleep she was, how much time she took to fall asleep again etc.

In another embodiment of the present invention, the third party application and/or data storage application may be configured observe the preferable conditions of sleeping of one or more users and give tips according to such conditions. For example if the time taken by a user to fall asleep in low illumination is less as compared to high illumination the third party application and/or data storage application may suggest the user to switch off the lights or dim the lights. Similarly, if the user cannot sleep because of low temperature, the third party application and/or data storage application may suggest the user to turn off the air conditioner.

The server computer can also display one or more information such as how a user sleeps as compared to the general guidelines of sleep, amount of sleep, daily rhythm, awake time in bed, or does the user really go to sleep or doing something else such as playing video games, stages of sleep, or how the user's sleep evolves over time, how child sleep patterns evolve from almost continuous sleep and no rhythm to day/night, how amount of sleep needed varies as a function of age etc.

In an embodiment of the present invention, the parent of a child user is allowed to observe a time history of the sleep data recording on the server computer. The access to the time history of sleep data may be password protected or may ask credentials of the user before allowing the observation of the sleep data. In some embodiments the data storage application and/or the third party application may exchange the sleep data with the calendar applications. The calendar application may arrange the sleep data according to e.g. customized time filters such as current month, year, days, hours and/or other limits.

The method is terminated at step 370.

It should be noted that any features, phases or parts of embodiment 30 can be freely permuted and combined with any of the embodiments 10, 20, 40, 50, 60, 70, 80, and/or 90 in a combination of two or more embodiments in accordance with the invention.

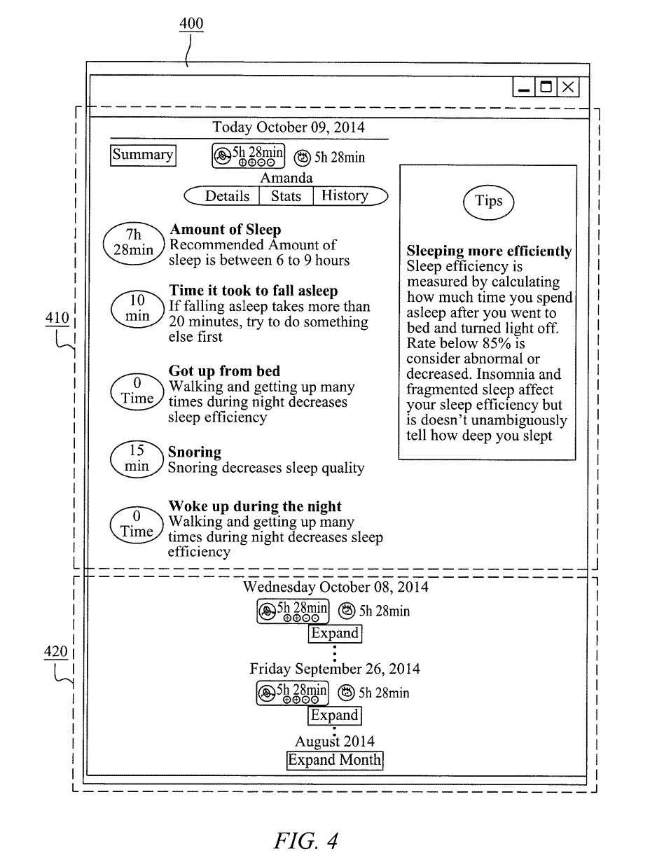

As depicted in FIG. 4, an embodiment 40 of the present invention provides a third party application to provide sleep data to the user. In the embodiment, the third party application 400 installed on the mobile subscriber terminal shows the amount of sleep 410 of each user with the related information for e.g. time of sleep, time taken to fall asleep, getting up time, snoring duration, how many times user got up from sleep in night and tips for efficient sleeping etc. In some embodiments the third party application 400 may also be a Internet browser, either as a separate application or as a part of the operating system of the mobile subscriber terminal.

The third party application 400 is configured to provide the time history of sleep 420 with the calendar application. The time history of sleep 420 can be customized to display e.g. months, weeks, years, and custom dates.

In this exemplary embodiment, the sleep data of Amanda is shown in date view of history of sleep 420. In the embodiment, the total sleep time of Amanda is given as seven hours and twenty-eight minutes. The distributed computer system 100 further shows that Amanda took 10 minutes to fall asleep and she did not get up from the bed while her sleep was interrupted two times in the night. The distributed computer system 100 further provides tips to Amanda for sleeping more efficiently. The sleep data shown in the date view can also contain comparisons to the user's past sleep measurements. For example, the mean or median sleep duration over a period of two weeks, one month, six months etc. is computed and compared to the sleep duration information in the date view. The comparison can be presented visually (with a bar chart etc.) or textually (“You slept 9 hours last night, which is above your 14-day median 8.1 hours”). In addition to sleep duration comparisons, heart rate, respiration rate, snoring amount etc. information can be compared in the date view.

It should be noted that any features, phases or parts of embodiment 40 can be freely permuted and combined with any of the embodiments 10, 20, 30, 50, 60, 70, 80, and/or 90 in a combination of two or more embodiments in accordance with the invention.

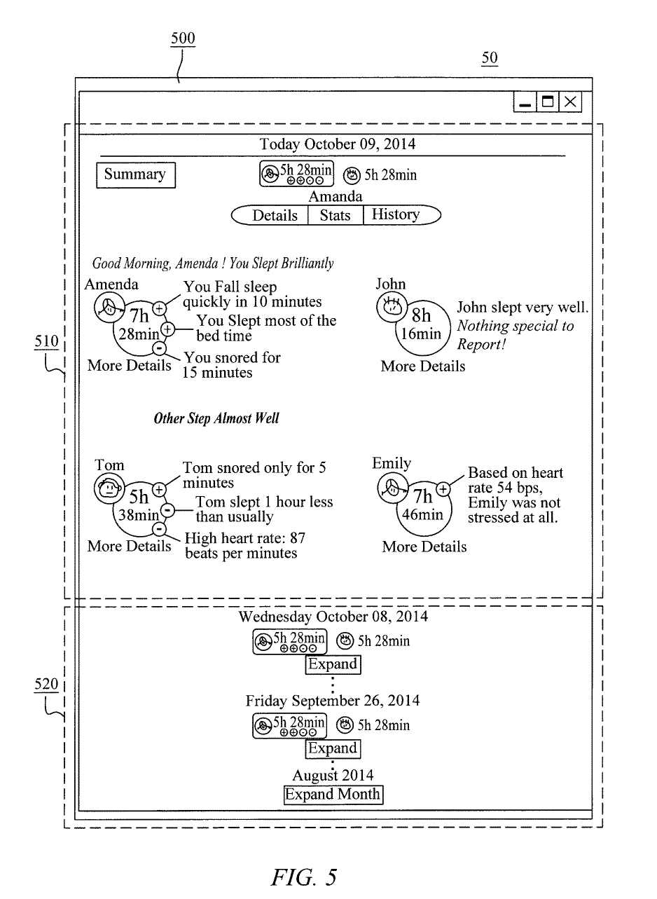

As demonstrated in FIG. 5 in embodiment 50 of the present invention, the third party application 500 is arranged to provide a sleep score 510 to each family member. The sleep score includes the time to fall asleep, sleeping time, snoring time, heart rate, stress measurement etc. The sleep score is to indicate that how well a user and/or the family of users sleep. The sleep scores of one or more users can be aggregated and can be customized according to the months, weeks, years, and custom dates. The sleeps scores 510 can be arranged in to a timeline 520 as shown. In the exemplary embodiment of the present invention, the sleep scores of Amanda, Tom, John and Emily are shown.

With this visualization, users can easily visualize the sleep details, for example, Amanda slept for 7 hours and 28 minutes, Tom slept for 5 hours and 38 minutes, John slept for 8 hours and 16 minutes and Emily slept for 7 hours and 46 minutes. Similarly, the snoring time, heart rate, and stress level is given for the users. It is also shown that Tom slept one hour less than his usual sleep time by comparing the current data with earlier record.

As illustrated above, the distributed computer system 100 can provide a display and/or user interface to one or more users for chronologically tracking the sleep score results of previous sleeps. The invention therefore enables even a lay person to track their night-to-night sleep performance in the home environment. With the help of the present invention people can make simple changes in their daily lifestyles according to the tips given by the third party application such as better physical activities and diet to improve sleep habits and to enhance their sleep performance. This may help decrease daytime tiredness and improve overall health and well-being.

It should be noted that any features, phases or parts of embodiment 50 can be freely permuted and combined with any of the embodiments 10, 20, 30, 40, 60, 70, 80, and/or 90 in a combination of two or more embodiments in accordance with the invention.

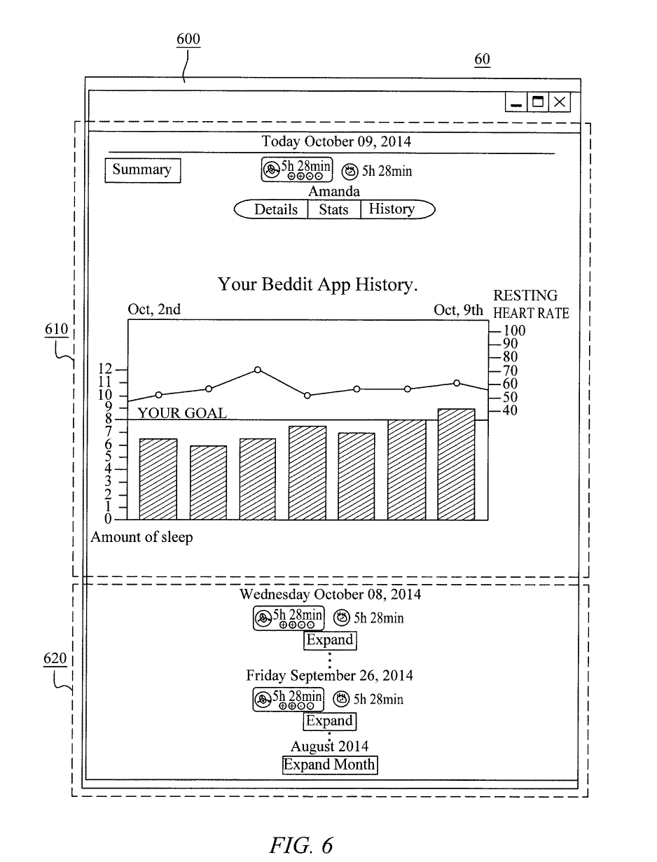

As demonstrated in FIG. 6, in an embodiment 60 of the present invention, the statistics of one user and the sleep data of the user are being analyzed against the user's past sleep data and other users' sleep data.

The third party application 600 is configured to provide the sleep data with the calendar application. The sleep data can be customized according to the months, weeks, years, and custom dates. In the embodiment of the invention, the third party applications installed at the mobile subscriber terminals of one or more family members are arranged to communicate and compare the sleep data of one or more family members. In the exemplary embodiment of the invention, the sleep data and the resting heart rate graph 610 of Amanda is provided for visualizing the sleep of a week. The third party application 600 provides an average sleep amount according to the age of user and the graph of sleep per night. It also provides the resting heart rate of the user for each sleep. From the figure, it is clear that Amanda's sleep and heart rate for the given week is not consistent. So the third party application 600 can give tips for the consistent sleep such as listen light music before sleep, eat light in night etc. In similar way, the demographics of other users can be visualized.

It should be noted that any features, phases or parts of embodiment 60 can be freely permuted and combined with any of the embodiments 10, 20, 30, 40, 50, 70, 80, and/or 90 in a combination of two or more embodiments in accordance with the invention.

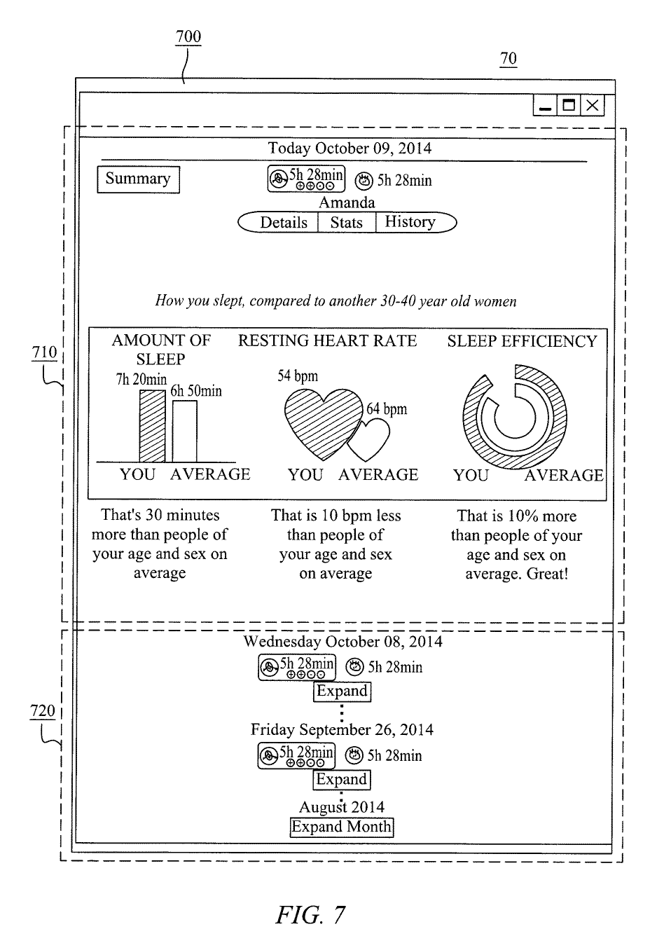

As demonstrated in FIG. 7, in an embodiment 70 of the present invention, the third party application 700 is configured to provide a comparison of the user sleep data with the general sleep data of a person from the user's age group. The third party application 700 is configured to provide the comparison in amount of sleep, resting heart rate, and sleep efficiency. In an exemplary embodiment, the sleep data of Amanda is being compared with the sleep data of a general thirty to forty year old woman. The peripheral device records the sleep, heart rate etc. and according to the recorded data the third party application indicates that the efficiency of sleep of the user. In addition to comparing based on the user's age and sex, the comparison can be performed based on e.g. the geographic location of users, the medical diagnosis of the users (e.g. a group could be formed from all users suffering from sleep apnea), or the profession of the users (e.g. a group could be formed from all lawyers in New York City).

In the exemplary embodiment, the third party application 700 indicates the amount sleep of Amanda as 7 hours and 28 minutes that is 30 minute more than the average sleep of persons of her age. Her heart is found 54 bpm that is good as compared to the average resting heart rate. Thus, the third party application 700 found that the Amanda sleeps 10% more effectively as compared to a general woman of her age.

In similar way, the third party application 700 can visualize the sleep efficiency of other users.

It should be noted that any features, phases or parts of embodiment 70 can be freely permuted and combined with any of the embodiments 10, 20, 30, 40, 50, 60, 80, and/or 90 in a combination of two or more embodiments in accordance with the invention.

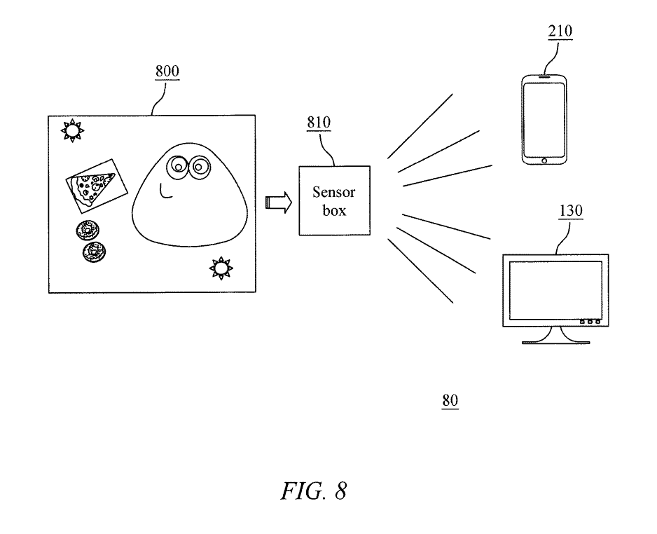

As demonstrated in FIG. 8, another embodiment 80 of the present invention provides a system for measuring sleep of a child user using a sensor box 810. It has been seen that the children have more affinity towards cartoon characters.

In the embodiment of present invention, the peripheral devices are arranged inside the sensor box 810. The sensor box 810 is covered or wrapped with a plastic or child friendly material sheet 800 having a favorite cartoon character of the child user embossed on it. The peripheral device senses the sleep data of the child user and communicates the data to the mobile subscriber terminal. The mobile subscriber terminal further communicates the data to the server 130 and/or the mobile subscriber terminal 210 of the child user's parent.

In some embodiments of the present invention, the sensor box 810 may be in the shape of a lunch box, pencil box and other child friendly objects having a plastic wrapper with cartoon characters embossed on it. In the embodiment of present invention, the Pou character is embossed on the sensor box 810. In another embodiment of present invention the sensor box 810 includes the peripheral devices and mobile subscriber terminal in it. The peripheral device includes a sleep sensor and is arranged to communicate the sleep data directly to the mobile subscriber terminal of the child user's parent and/or to the data storage application of the server computer 130.

Further, in another embodiment of the present invention, the sensor box 810 can be in the form of a very thin sheet of fabric having a peripheral device and/or the mobile subscriber terminal incorporated therein. The thin sheet can be placed under the child user's bed or mattress. Further, the sensor box 810 can be in the form of a drawing sheet and/or storybook of the child user that the child user likes to keep with him while sleeping. The sensor box 810 in some embodiments of the present invention can be equipped with one or more of a camera, audio-visual alert devices, music devices, speakers, and other device that may help the child user to fall asleep.

In some aspects of the present invention, the peripheral device includes one or more sensors for sensing the sleep data. The peripheral device can include one or more sensors including but not limited to the capacitive sensor, inductive sensor, light sensor, humidity sensor, proximity sensors and any other sensors now known or later developed. In some aspects of the present invention, the mobile subscriber terminal may be any device for e.g. a laptop, tablet, smart phone, and computer, ASIC and any other device capable of executing the third party applications. In an embodiment of present invention, the sensor box 810 can be configured in a way that it may have the display of third party subscriber terminal arranged on the top and/or on the side surfaces of the sensor box for visualizing the sleep data. In other embodiments, the sensor box 810 can have an animated and/or cartoon figured lid or flap cover for cover up the display of the mobile subscriber terminal.

It should be noted that any features, phases or parts of embodiment 80 can be freely permuted and combined with any of the embodiments 10, 20, 30, 40, 50, 60, 70, and/or 90 in a combination of two or more embodiments in accordance with the invention.

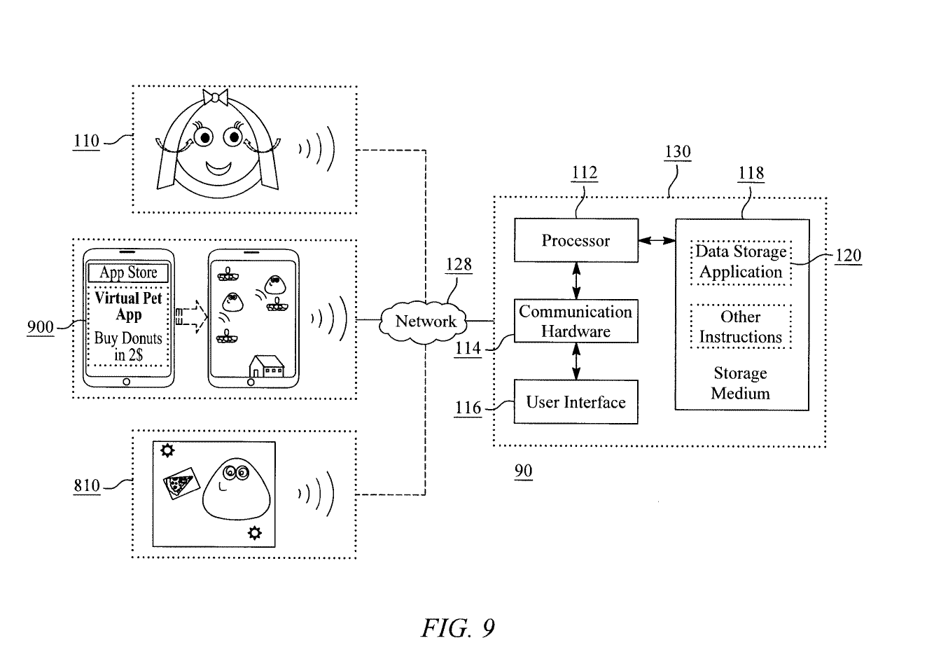

As depicted in FIG. 9, in another embodiment 90 of the present invention, a virtual pet application 900 is stored on the mobile subscriber terminal of the child user.

In the embodiment 90, the computer peripheral is realized in a soft toy for a child and/or the third party software application is the virtual pet software application 900 that acts as a game for the child user and allows for in-app purchases. The virtual pet application 900 is configured to motivate children to monitor their sleep, and to stay within sleep guidelines and/or limits set by parent. This is achieved e.g. by giving rewards for e.g. in virtual currency to buy items for the virtual pet as rewards for following the sleeping times and behavior required by the parent.

For example, in one embodiment the child user is enabled to play with virtual pet as he plays with the real pet. The virtual pet application 900 is configured to give rewards, on getting to bed early enough, following evening routines, starting sleeping early, and waking up on time etc.

This virtual pet application 900 will help the child user in improving the sleep habits and hence affect their behaviour positively in day time. The virtual pet application 900 can be purchased from any application store. The virtual pet application 900 in an embodiment of the present invention can be configured to communicate with the third party application and also with other virtual pet applications installed on other mobile subscriber terminals, e.g. in a social networking manner. It may share the reward data with the virtual pet application 900 and/or the other applications installed on the other child users' mobile subscriber terminals to encourage them to sleep properly.

In one aspect of the present invention, the virtual pet of the virtual pet application 900 can be enabled to feel like a real pet. The virtual pet can respond to the child user with audio and/or visual effects. The virtual pet can also be trained according to one or more data sets to respond on the activities of child user in order to attract the child user.

In one aspect of the present invention, the virtual pet application 900 is configured to work with a mobile subscriber terminal and/or a peripheral to monitor the sleep. The peripheral device used by the child user will sense the sleep data and communicate the sleep data to the mobile subscriber terminal. The mobile subscriber terminal is arranged to communicate the sleep data to the server computer 130 and/or a parent mobile subscriber terminal 210. The virtual pet application 900 will give rewards to the child user if the sleep behaviour of child user is found to be in order. The rewards may be in the form of virtual currency that may allow the child to make an in-application purchase of donuts, fruits, candies and/or other fascinating objects appealing to the child.

In another aspect of the present invention, the virtual pet application 900 is configured to communicate with the soft toy application of the soft toy. The soft toy application will communicate the sleep data to the virtual pet application 900 in order to provide the rewards to the child user for sleeping properly. In the embodiment of the present invention, the child user may invite his friends and/or siblings to play with him on the virtual pet application. The parent user can also monitor such invitations for security and privacy purposes.

It should be noted that any features, phases or parts of embodiment 90 can be freely permuted and combined with any of the embodiments 10, 20, 30, 40, 50, 60, 70, and/or 80 in a combination of two or more embodiments in accordance with the invention.

In an aspect of the present invention, the sleep data recordings on said server computer are arranged to measure and/or display including but not limited to one or more of: how user's sleep compares to general guidelines, amount of sleep, daily rhythm, awake time in bed, does user really go to sleep, or e.g. play mobile games in the room, deep/light/active sleep stages, follow how user's sleep evolves over time, how child user sleep patterns evolve from almost continuous sleep and no rhythm to day/night rhythm, and how amount of sleep needed varies as a function of age etc.

Further, a plurality of peripheral devices are each arranged to measure each family member, and the sleep data from all measured family members is arranged to be aggregated to said server computer, and the aggregate data is arranged to be displayed together to visualize how the sleeping behaviors of the family members influence each other.

Some embodiments of the present invention are configured to invite one or more users to the distributed system via an email invite, application message or a social networking friend request. Further, the distributed computer system 100 allows at least one of the parents to see all Beddit devices at home, and the user can assign other users in third party applications and other applications. The distributed system is further configured to show an invite request in a virtual pet application and the system may ask a child user if he knows the person who sent the invitation.

Embodiments of the present invention provide the child user with a possibility to measure sleep, further to communicate said sleep data and further to encourage the child user to maintain healthy sleeping habits in an administratively efficient and fun manner.

It should be noted that any features, phases or parts of any embodiment 10, 20, 30, 40, 50, 60, 70, 80, and/or 90 can be freely permuted and combined with any of the embodiments 10, 20, 30, 40, 50, 60, 70, 80 and/or 90 in a combination of two or more embodiments in accordance with the invention.

Some or all of the aforementioned advantages of the invention are accrued by a distributed computer system 100 for tracking sleep of all family members by providing a soft toy and/or a virtual pet to children and wellness information to adults using the cloud based data to improve the health and wellness over time, and hence change sleeping behavior of one or more users.

In addition and with reference to the aforementioned advantage accruing embodiments, the best mode of the invention is considered to be where the peripheral device is realized in a soft toy for a child and/or the third party software application is a virtual pet software application that allows for in app purchases. The soft toy is arranged to measure the sleep of at least one child user and in which the sleep data of the child user can be seen by turning the eyes of the soft toy. In one alternative embodiment the eyes of the soft toy turn red if the sleep score is bad, and appear natural blue or brown if the sleep score is good.

Further advantages and features of the present invention are considered by configuring the distributed system to follow: how children's sleep evolves over time (in analogous way that growth charts track weight and height); how child's sleep patterns evolve from almost continuous sleep and no rhythm to day/night rhythm; how amount of sleep needed decreases; but continues to be higher than for adults; how in kindergarten/school child is required to wake up at a specified time; does child still get enough sleep; how easy it is to fall asleep at evening, and wake up in the morning; see if anyone in the family has issues with their sleep; how one family member's sleep affects the other family members; is family members sleep normal compared to recommendations; is family members sleep normal compared to age group and sex, when at elderly age sleep patterns start again to change and get instructions for improving situation. The distributed computer system 100 enables the user to visualize the sleep data of a long time frame such as of years, months, weeks and days.

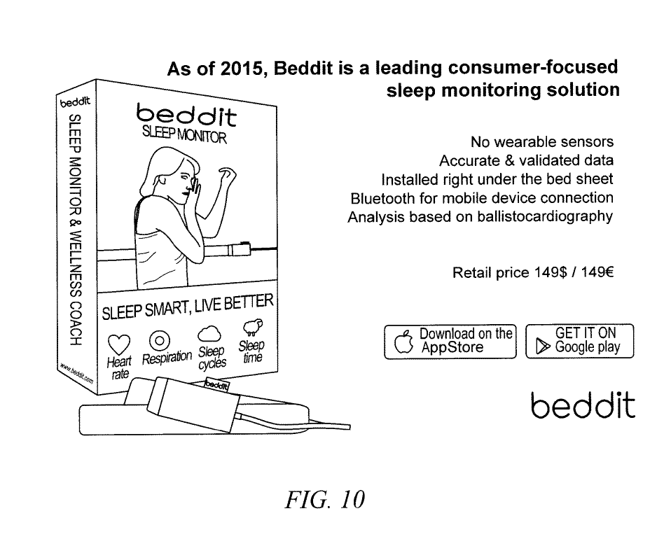

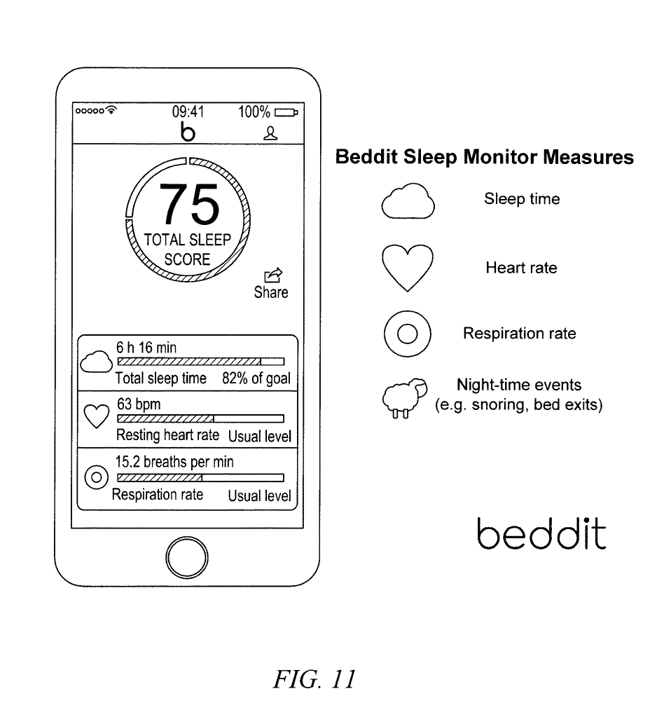

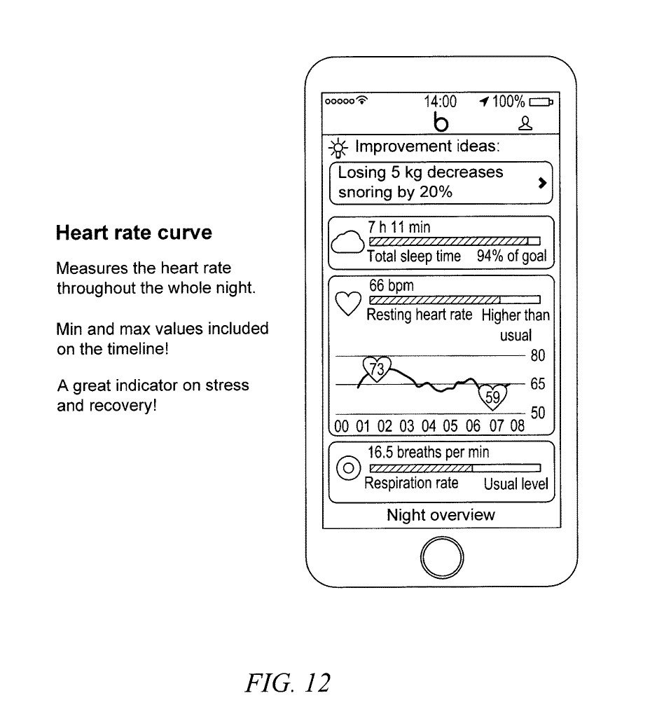

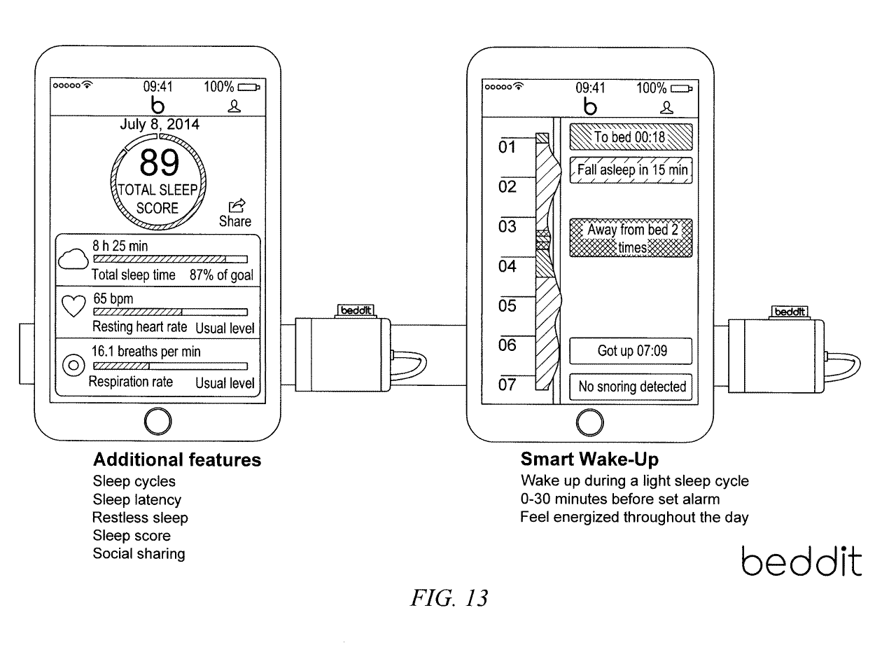

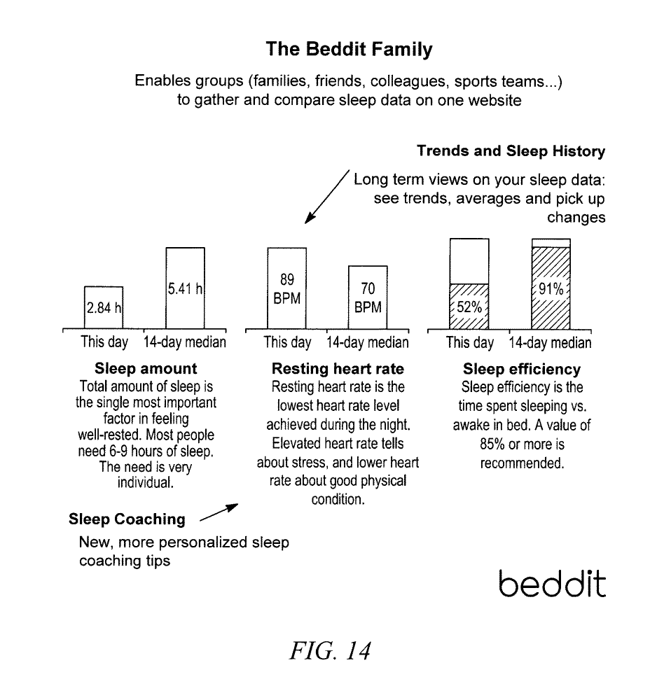

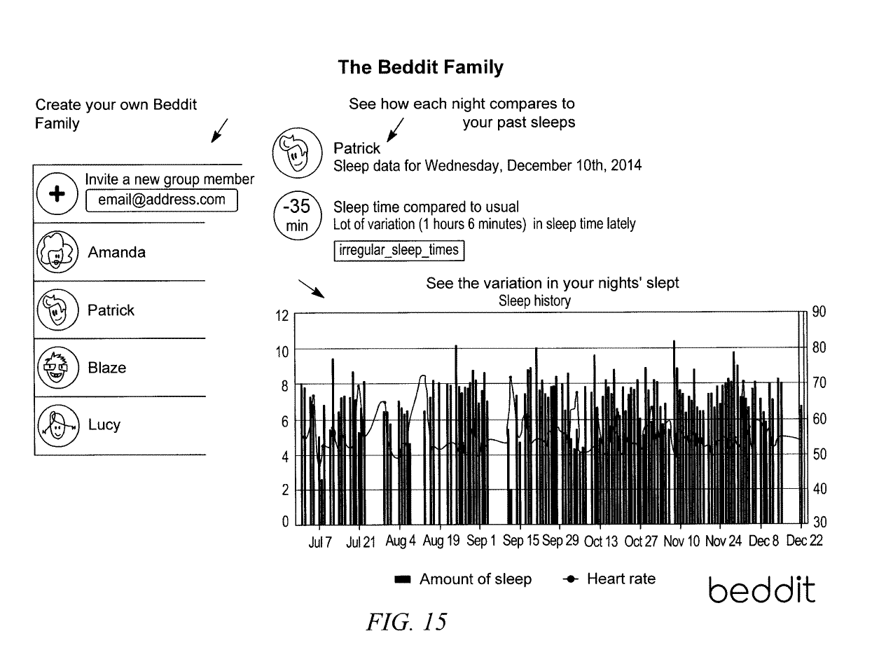

FIGS. 10-15 comprise description of an actual product based on the invention, which will soon be launched after the filing of the present application.

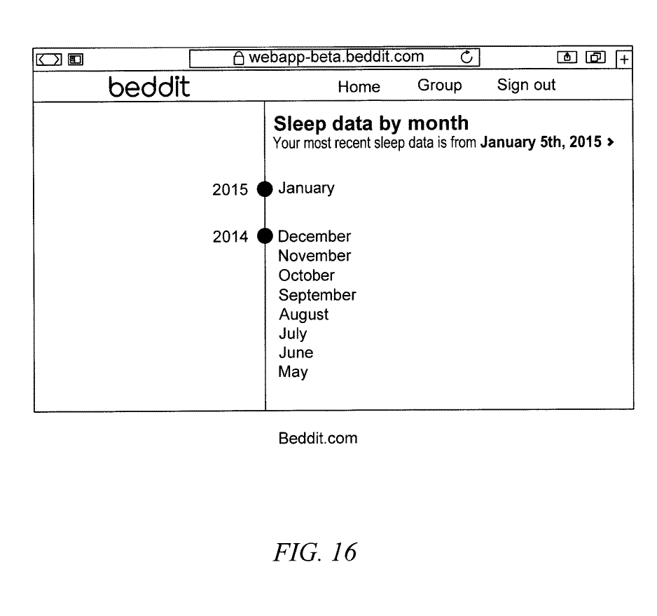

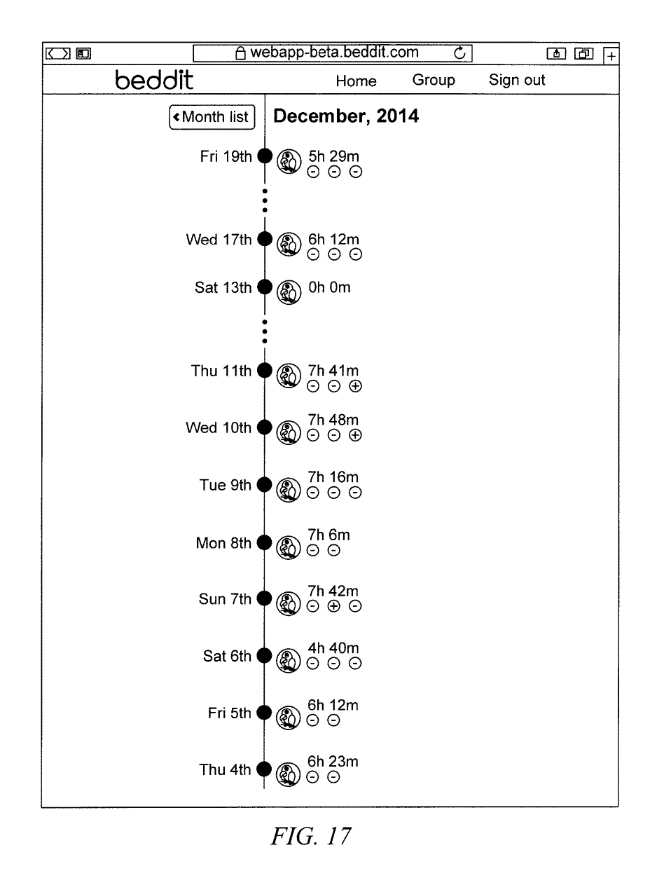

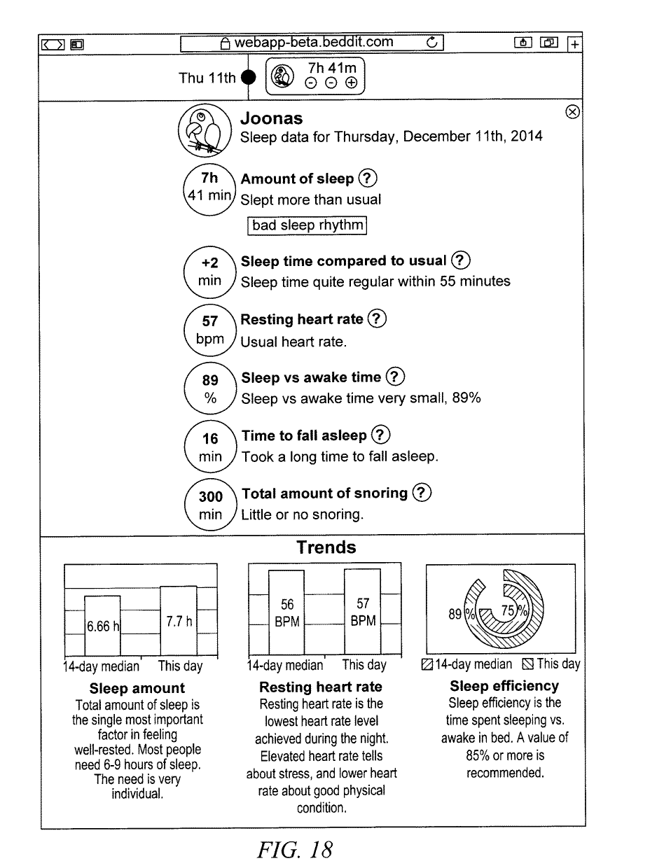

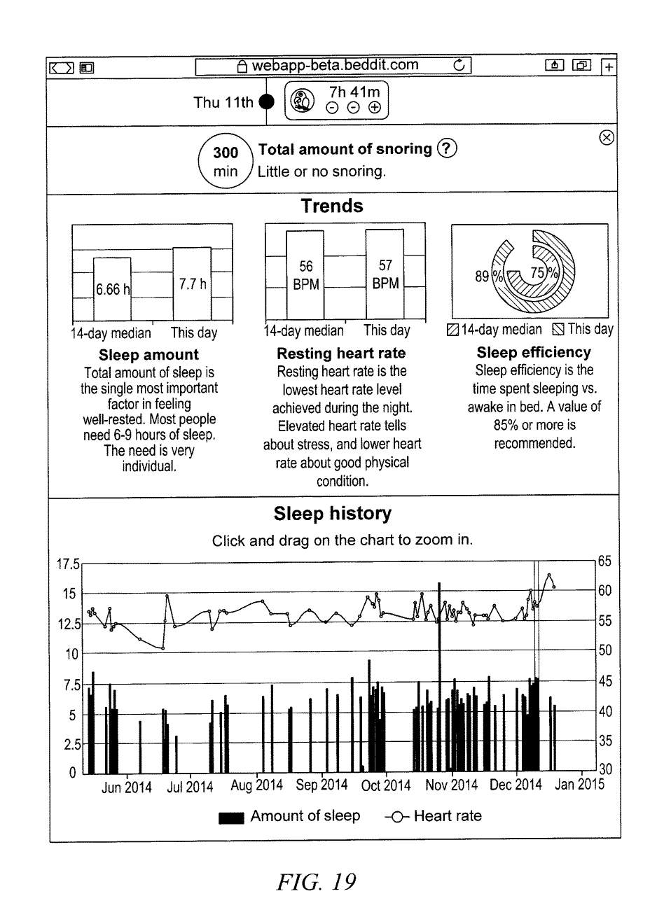

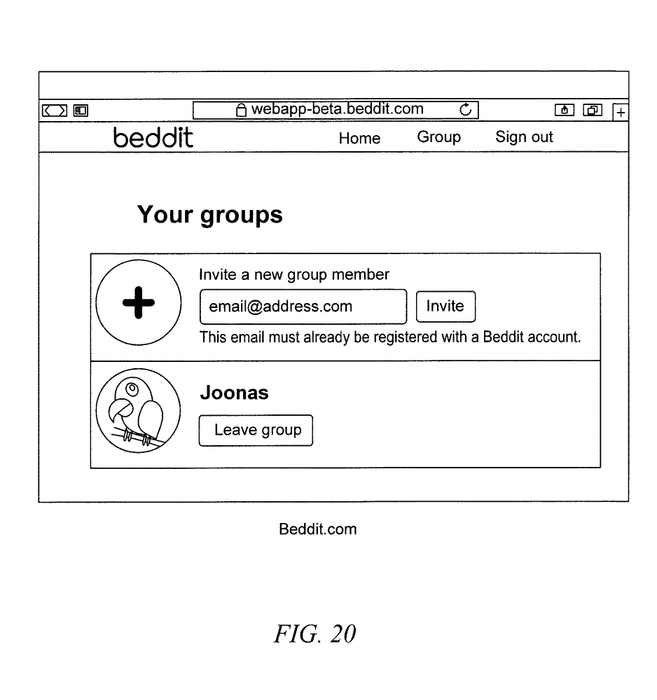

FIGS. 16-20 comprise screenshots of the actual product in secret beta-testing phase.

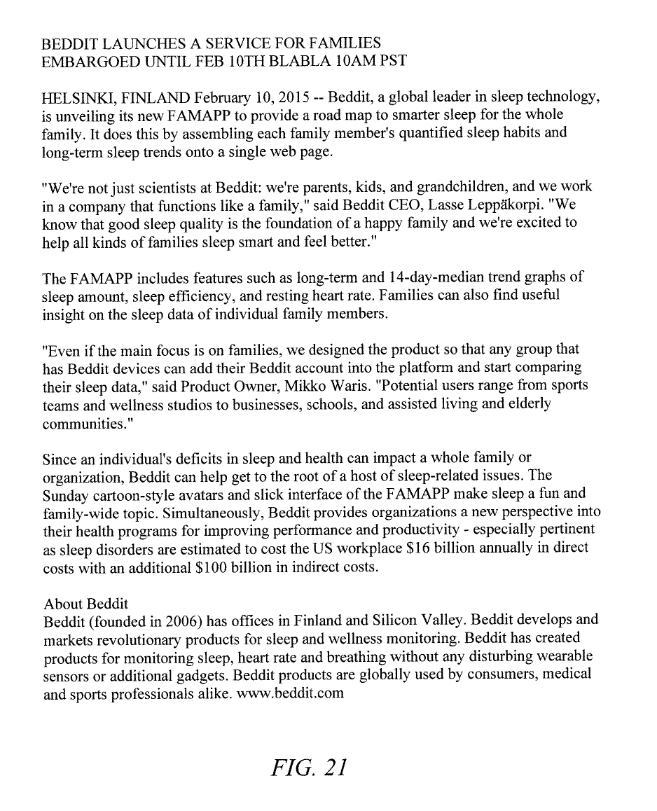

FIG. 21 demonstrates the unpublished press release describing one embodiment of the product of the invention. It has a text as follows:

Beddit Launches a Service for Families

Embargoed Until February 10th Blabla 10 AM PST

HELSINKI, FINLAND Feb. 10, 2015—Beddit, a global leader in sleep technology, is unveiling its new FAMAPP to provide a road map to smarter sleep for the whole family. It does this by assembling each family member's quantified sleep habits and long-term sleep trends onto a single web page.

“We're not just scientists at Beddit: we're parents, kids, and grandchildren, and we work in a company that functions like a family,” said Beddit CEO, Lasse Leppäkorpi. “We know that good sleep quality is the foundation of a happy family and we're excited to help all kinds of families sleep smart and feel better.”

The FAMAPP includes features such as long-term and 14-day-median trend graphs of sleep amount, sleep efficiency, and resting heart rate. Families can also find useful insight on the sleep data of individual family members.

“Even if the main focus is on families, we designed the product so that any group that has Beddit devices can add their Beddit account into the platform and start comparing their sleep data,” said Product Owner, Mikko Waris. “Potential users range from sports teams and wellness studios to businesses, schools, and assisted living and elderly communities.”

Since an individual's deficits in sleep and health can impact a whole family or organization, Beddit can help get to the root of a host of sleep-related issues. The Sunday cartoon-style avatars and slick interface of the FAMAPP make sleep a fun and family-wide topic. Simultaneously, Beddit provides organizations a new perspective into their health programs for improving performance and productivity—especially pertinent as sleep disorders are estimated to cost the US workplace $16 billion annually in direct costs with an additional $100 billion in indirect costs.

About Beddit

Beddit (founded in 2006) has offices in Finland and Silicon Valley. Beddit develops and markets revolutionary products for sleep and wellness monitoring. Beddit has created products for monitoring sleep, heart rate and breathing without any disturbing wearable sensors or additional gadgets. Beddit products are globally used by consumers, medical and sports professionals alike. www.beddit.com

The subject matter of the drawings 10-21 is believed to overlap with the subject matter explained in association with FIGS. 1-9. However, It should be noted that any features, phases or parts of any embodiment 10, 20, 30, 40, 50, 60, 70, 80 90 and/or those of FIGS. 10-21 can be freely permuted and combined with any of the embodiments 10, 20, 30, 40, 50, 60, 70, 80, 90 and/or those of FIGS. 10-21 in a combination of two or more embodiments in accordance with the invention.

Further, it should be noted that the names and articles used herein are only for the indicative purposes and for the understanding of the person skilled in the art. For example, the parent may be any care taker, guardian, nurse and any other person. Similarly, the child user can be an infant, a mentally impaired adult or any sick person that needs sleep monitoring.

The invention has been explained above with reference to the aforementioned embodiments and several commercial and industrial advantages have been demonstrated. The methods and arrangements of the invention allow the measurement of sleep using a sensor device and a mobile third party client application for the whole family. This reduces the administrative burden borne in following the sleeping habits of family members as a group significantly.

The invention has been explained above with reference to the aforementioned embodiments. However, it is clear that the invention is not only restricted to these embodiments, but includes all possible embodiments within the spirit and scope of the inventive thought and the following patent claims.

1. A system for determining sleep of at least one user, the system comprising:
a plurality of devices including:
a first device configured to:
measure a first sleep data of a first user, the first user included in a plurality of users, and
generate a first signal indicative of the first sleep data, the first signal included in a plurality of signals; and
a second device configured to:
measure a second sleep data of a second user, the second user included in the plurality of users, and
generate a second signal indicative of the second sleep data, the second signal included in the plurality of signals;
at least one mobile subscriber terminal configured to receive at least one of the plurality of signals from one of the plurality of devices;
a network configured to receive the plurality of signals from the at least one mobile subscriber terminal; and
a computer including:
a processor configured to:
receive the plurality of signals from the network,
aggregate the plurality of signals,
determine an influence of sleep behavior of one of the plurality of users to others using the aggregated plurality of signals.

2. The system of claim 1, wherein each mobile subscriber terminal includes a third party application configured to determine a time history associated with the respective sleep data from the calendar application.

3.-4. (canceled)

5. The system of claim 1, wherein the at least one mobile subscriber terminal is included in a plurality of mobile subscriber terminals, and
wherein at least two of the plurality of mobile subscriber terminals include one or more applications and are configured to communicate with each other using the one or more applications.

6. The system of claim 1, wherein at least one of the plurality of devices is a toy, the toy includes:
a plurality of eyes configured to display information associated with the respective sleep data.

7.-10. (canceled)

11. The system of claim 1, wherein at least one the plurality of devices is a sensor box.

12. (canceled)

13. The system of claim 1, wherein at least one mobile subscriber terminal is included in a plurality of mobile subscriber terminals, each device is configured to communicate with one of the plurality of mobile subscriber terminals.

14.-15. (canceled)

16. A method for recording sleep data of plurality of users, the method comprising:
initiating a first communication connection between a first peripheral device and a server computer;
measuring a first sleep of a first user using the first peripheral device, the first user included in the plurality of users;
generating a first signal indicative of the first sleep, the first signal including a first sleep data, the first sleep data included in the sleep data;
initiating a second communication connection between a second peripheral device and the server computer;
measuring a second sleep of a second user using the second peripheral device, the second user included in the plurality of users;
generating a second signal indicative of the second sleep, the second signal including a second sleep data, the second sleep data included in the sleep data;
aggregating the sleep data of the plurality of users; and
displaying the aggregated sleep data.

17. The method of claim 16, further comprising:
determining a time history associated with the respective sleep data from a calendar application included in a mobile subscriber terminal associated with the respective peripheral device.

18. The method of claim 16, further comprising:
recording a weight and a height of the first user over a plurality of time periods;
storing the first sleep data; and
determining a relationship between the first sleep data and the weight and the height of the first user.

19.-20. (canceled)

21. The method of claim 16, wherein the first peripheral device is a toy, the method further comprising:
displaying information associated with the first data using eyes of the toy.

22.-45. (canceled)

46. The system of claim 1, wherein the first device includes a first sensor configured to:
detect a first type of movement, the first type of movement associated with a user's weight, and
generate a first signal associated with the detected first type of movement.

47. The system of claim 46, wherein the first sensor includes one or more of a piezoelectric sensor and a piezo thin film sensor.

48. The system of claim 1, wherein the first device includes a second sensor configured to:
detect a second type of movement, the second type of movement associated with the at least one user moving while on a bed, and
generate a second signal associated with the detected second type of movement.

49. The system of claim 48, wherein the second sensor includes one or more of a strain gauge, a capacitive sensor, a proximity sensor, a thermocouple, and an inductive sensor.

50. The method of claim 16, wherein initiating the first communication connection between the first peripheral device and the server computer includes:
communicating the first sleep data from the first peripheral device to a first mobile subscriber terminal; and
communicating the first sleep data from the first mobile subscriber terminal to the server computer.

51. The method of claim 50, wherein communicating the first sleep data from the first mobile subscriber terminal to the server computer includes:
communicating the first sleep data from the first mobile subscriber terminal to a network; and
communicating the first sleep data from the network to the server computer.

52. The method of claim 16, wherein generating the first signal indicative of the first sleep includes:
analyzing the measured first sleep of the first user using an application associated with a first mobile subscriber terminal,
wherein the sleep data is aggregated using the same application or another application associated with the first mobile subscriber terminal.

53. The method of claim 16, wherein initiating the first communication connection between the first peripheral device and the server computer includes:
communicating the first sleep data directly from the first peripheral device to the server computer.

54. The method of claim 16, further comprising:
analyzing a sleep quality of the first user based on the first sleep data; and
providing suggestions on how to improve sleep using the analyzed sleep quality.
